(19) United States
(12) Patent Application Publication
(10) Pub. No.: US 2021/0252213 A1
(43) Pub. Date: Aug. 19, 2021

(54) IRONLESS ELECTRIC MOTOR FOR MRI COMPATIBILITY

(71) Applicant: KONINKLIJKE PHILIPS N.V., EINDHOVEN (NL)

(72) Inventors: Gerard Johannes Pieter NIJSSE, Bodegraven (NL); Funda SAHIN NOMALER, Eindhoven (NL); Wouter KLOP, Eindhoven (NL); Aditya MEHENDALE, Waalre (NL); Petrus Carolus Maria FRISSEN, Beek (NL); Olav Johannes SEIJGER, Eindhoven (NL)

(21) Appl. No.: 17/254,921

(22) PCT Filed: Jun. 26, 2019
(86) PCT No.: PCT/EP2019/066922

§ 371 Date: Dec. 22, 2020

Related U.S. Application Data
(60) Provisional application No. 62/691,955, filed on Jun. 29, 2018.

(51) Int. Cl. A61M 5/145; H02K 1/12; H02K 3/47; A61B 5/055; A61B 5/00

(52) U.S. Cl. CPC A61M 5/1452; H02K 1/12; A61B 5/0036; A61B 5/055; A61B 5/4839; H02K 3/47

(57) ABSTRACT

An electric motor (20) usable in proximity to a magnetic resonance imaging (MRI) device (4) includes a stator (30) comprising electrical windings (32), and a rotor (40, 50, 60) magnetically coupled with the stator. The electric motor does not include ferromagnetic material, and the electric motor does not include any permanent magnet. The rotor may include an outer rotor cylinder (50, 60) surrounding the stator, and may further include an inner rotor cylinder (40) disposed inside the stator and connected to rotate with the outer rotor cylinder. The rotor may comprise a cylindrical sheet rotor (40, 50). Alternatively, the rotor (60) may comprise one or more conductive loops (62A, 62B, 62C) each shaped such that the induced voltage in one loop portion (HL1) cancels the effect of the induced voltage in another loop portion (HL2), and a coupled split stator (301, 302). In another disclosed aspect, an infusion pump (10) includes the electric motor.

### FIELD

The following relates generally to the medical device arts, infusion pump arts, magnetic resonance imaging (MRI) arts, electric motor arts, and related arts.

### BACKGROUND

Magnetic resonance imaging (MRI) is a powerful medical diagnostic and clinical assessment technique. However, MRI generates strong magnetic fields and radio frequency (RF) interference, and in turn MRI images are susceptible to degradation due to RF interference from nearby magnetic fields and/or RF emitting devices. In view of this, medical MRI systems are generally enclosed in an RF shielded room (sometimes referred to as the MRI room), that is, a room in which the walls (and possibly floor and/or ceiling) include a wire mesh sheeting or the like forming an enclosing Faraday cage. Patients undergoing an MRI examination procedure are evaluated pre-procedure to ensure they do not have excessive implanted ferromagnetic material—for example, any implanted cardiac pacemaker is required to be MRI compliant or MRI safe. Laboratory safety protocols prohibit items containing ferromagnetic materials. In general, it is prohibited to introduce or use ferromagnetic materials in the MRI room because the MRI field may cause large attraction forces, leading to dangerous situations, and because the ferromagnetic material may distort the MRI system's imaging.

This situation creates difficulties for using motorized devices such as infusion pumps, fans, motorized patient tables, or the like in an MRI room. An electric motor is an electromagnetic device, and employs interaction between electric and magnetic fields to convert input electrical power into motive (mechanical) force output, usually in the form of a rotating shaft whose rotation is driven by the motor. In such motors, windings are wrapped around a ferromagnetic core to form an electromagnet producing the magnetic field when the coil is electrically energized. These are arranged as stator windings which are mounted in a stationary fashion, and rotor windings mounted on a rotating element (rotor). Interaction between the stator and rotor magnetic fields produces the motive force. Alternatively, one of these magnetic fields may be provided by a permanent magnet comprising magnetized ferromagnetic material. In an induction motor, only one set of windings (usually the stator windings) is electrically energized using an input alternating current (a.c. current), and the resulting time-varying magnetic field induces a.c. current in the rotor windings thereby providing the interacting magnetic field generating the motive force on the rotor. An induction motor thus operates in a fashion akin to a transformer, except that the output is rotation of the secondary electromagnet in an induction motor, rather than the electrical current induced in the secondary electromagnet. In a variant inductor motor design, the rotor windings are replaced by short-circuited electrically conductive bars—this is referred to as a squirrel cage rotor.

Such motors are problematic when used in an MRI room. The ferromagnetic material presents a physical hazard if it is drawn into the MRI bore by the intense magnetic field generated by the MRI device. Furthermore, both the ferromagnetic material and the generated magnetic fields can interfere with operation of the MRI device, thereby leading to degraded clinical MRI images and potential for medical misdiagnosis.

Various approaches are employed to address the difficulty of using an electric motor in an MRI room. These approaches generally require employing a specially designed motor that is MRI compatible. For example, an electrostatic motor operating on the basis of attraction and repulsion of electric charge can be employed. However, electrostatic motors are a non-standard motor design, and generally require high operating voltages and provide low efficiency, and are more typically used for miniaturized devices, e.g. micro-electro-mechanical systems (MEMS). The high voltages can also introduce electrostatic discharges with concomitant RF noise. Piezoelectric motors have similar difficulties. Another approach is to locate the electric motor outside the MRI room and run the rotating shaft through the wall into the MRI room. This approach requires a long rotating shaft, complicates operation as the motor is located outside of the MRI room, and the shaft penetration compromises integrity of the RF shielding of the MRI room. In the case of dedicated devices that are used only in the MRI room, specialized motor designs are known that make use of the magnetic field generated by the MRI device itself in the motor operation. See, e.g. Roeck et al., U.S. Pub. No. 2010/0264918 A1. Such a motor is only usable inside the MRI room due to its reliance on the magnetic field generated by the MRI device. This means the infusion pump cannot go with the patient to and from the MRI room, which presents substantial practical difficulties.

While operation in the MRI room, or in proximity to an MRI device, is an illustrative problem, there are other situations in which an electric motor can be problematic due to potential for detrimental magnetic interactions. For example, in positron emission tomography (PET) imaging, photomultiplier tube (PMT)-based radiation detectors are susceptible to magnetic interference. Electric motors in proximity to sensitive magnetometer devices such as superconducting quantum interference device (SQUID) devices can lead to erroneous magnetic field measurements. These are merely illustrative examples.

The following discloses a new and improved systems and methods.

### SUMMARY

In one disclosed aspect, an electric motor includes a stator comprising electrical windings, and a rotor magnetically coupled with the stator. The electric motor does not include ferromagnetic material, and the electric motor does not include any permanent magnet. The rotor optionally includes an outer rotor cylinder surrounding the stator. The rotor optionally further includes an inner rotor cylinder disposed inside the stator and connected to rotate with the outer rotor cylinder. The outer rotor cylinder may comprise a cylindrical sheet rotor. The electrical windings of the stator are, in illustrative embodiments, wound to form the stator as a three-phase stator. The electric motor may further comprise a fixed frequency motor driver operative to electrically power the stator at a fixed electrical frequency.

In another disclosed aspect, an infusion pump comprises an electric motor as set forth in the immediately preceding paragraph, along with a fluid delivery component comprising one of (i) a syringe receptacle and (ii) a fluid pump having an inlet configured to connect with an infusion fluid supply and an outlet configured to connect with a patient infusion delivery accessory. The electric motor is connected to operate the fluid delivery component by driving a plunger of an associated syringe mounted in the syringe receptacle or by operating the fluid pump.

In another disclosed aspect, a method of operating a medical device is disclosed. The method comprises operatively connecting the medical device to a patient, and operating an electric motor to apply motive force to the medical device to deliver a therapy to the patient. The electric motor does not include ferromagnetic material and does not include a permanent magnet.

One advantage resides in providing an electric motor with no ferromagnetic material.

Another advantage resides in providing an electric motor with no ferromagnetic material and no permanent magnet.

Another advantage resides in providing an electric motor which is compatible with an MRI device and with use inside an MRI room.

Another advantage resides in providing an electric motor with one or more of the foregoing benefits which retains a conventional induction motor design.

Another advantage resides in providing an electric motor with one or more of the foregoing benefits which retains a conventional induction motor design with a reduced number of component and/or reduced manufacturing cost.

Another advantage resides in providing an electric motor with one or more of the foregoing benefits which further provides intrinsic RF shielding.

Another advantage resides in providing an electric motor with one or more of the foregoing benefits which is further operable using a fixed frequency motor driver operative to electrically power the stator at a fixed electrical frequency to operate the electric motor.

Another advantage resides in providing an MRI-compatible infusion pump employing an electric motor with one or more of the foregoing benefits.

Another advantage resides in providing an electric motor or an MRI-compatible infusion pump employing such an electric motor, which is MRI-compatible but also usable outside of the MRI room and not in proximity to the MRI device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates an illustrative electric motor application setting including an MRI room containing an MRI device, in which an infusion pump employs an electric motor as disclosed herein.

FIGS. 2 and 3 diagrammatically illustrate an electric motor comprising an induction motor which does not include ferromagnetic material and does not include any permanent magnet, according to one illustrative embodiment, where FIG. 3 shows a diagrammatic side view of the electric motor and FIG. 2 shows Section A-A indicated in FIG. 3.

FIGS. 4 and 5 diagrammatically illustrate an electric motor comprising an induction motor which does not include ferromagnetic material and does not include any permanent magnet, according to another illustrative embodiment, where FIG. 5 shows a diagrammatic side view of the electric motor and FIG. 4 shows Section B-B indicated in FIG. 5.

FIGS. 6 and 7 diagrammatically illustrate an electric motor comprising an induction motor which does not include ferromagnetic material and does not include any permanent magnet, according to another illustrative embodiment, where FIG. 7 shows a diagrammatic side view of the electric motor and FIG. 6 shows Section C-C indicated in FIG. 7.

FIGS. 8, 9, and 10 plot calculated motor characteristics for an electric motor comprising an induction motor which does not include ferromagnetic material and does not include any permanent magnet, according to calculations as described herein.

FIG. 11 diagrammatically illustrates a perspective view of an alternative rotor comprising windings in a double loop pattern, along with a split stator diagrammatically indicated by dashed lines.

FIG. 12 diagrammatically illustrates a perspective view of a variant of the rotor/stator design of FIG. 11 in which the rotor windings are electrically energized via commutators.

### DETAILED DESCRIPTION

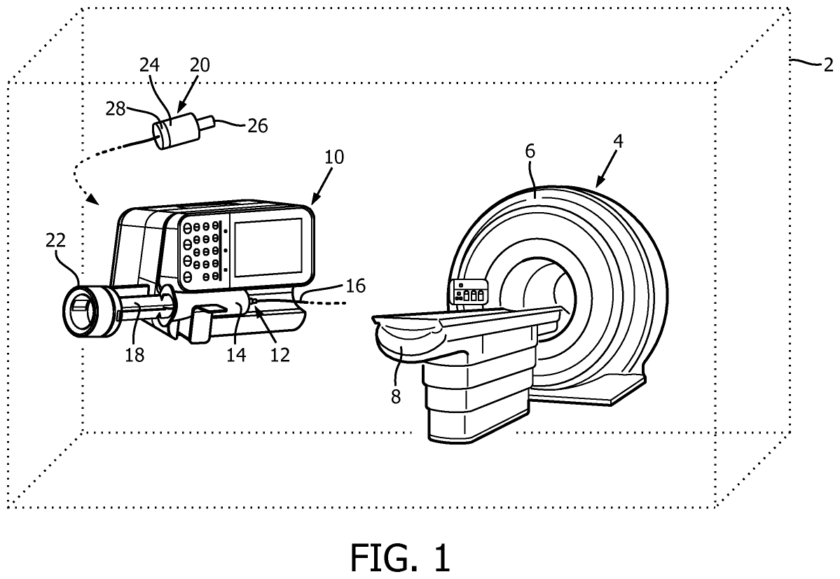

With reference to FIG. 1, an illustrative electric motor application setting is shown, including an MRI room 2 containing an MRI device 4 which includes a housing 6 containing a magnet generating a substantial magnetic field. For example, the illustrative MRI device 4 may be a Philips Achieva™ 1.5T MRI device in which the magnet generates a static magnetic field (sometimes referred to as the Bo magnetic field) of about 1.5 Tesla. Other MRI devices for clinical applications available from Philips or other manufacturers typically generate Bo fields on the order of 0.2-7.0 Tesla, although lower or higher main magnetic field strength is also contemplated. The MRI housing 6 typically also contains magnetic field gradient coils that superimpose spatially varying magnetic field gradients on the Bo magnetic field for purposes such as spatially selective magnetic resonance excitation, spatially encoding the phases and/or frequencies of excited magnetic resonances, spoiling magnetic resonances, and/or other purposes. An illustrative patient support 8 is provided for loading a patient into the MRI device 4 for imaging and for withdrawing the patient after completion of the MRI imaging session, and may also provide for other adjustments such as moving the patient stepwise through the MRI device to acquire a series of MRI images forming a “whole body” scan. Although not illustrated, the MRI device 4 typically includes other conventional MRI components such as a whole-body RF coil and/or local RF coils for exciting and/or detecting magnetic resonances, electronics for energizing the gradient coils, RF coils, or so forth, a cryogenic compressor for maintaining the magnet at cryogenic temperature (in cases where the MRI magnet is a superconducting magnet), and/or so forth.

The patient may require medical assistance or therapy during the MRI imaging procedure. For example, an infusion pump 10 may be employed to deliver an infusion fluid to the patient, e.g. a saline solution, an infused medication, or so forth. The illustrative infusion pump 10 is a syringe infusion pump including a syringe receptacle 12 into which a syringe 14 is inserted. (It is also noted that FIG. 1 is not to scale, e.g. relative sizes of the illustrations of the MRI device 4 and infusion pump 10, respectively, are not to scale). A patient infusion delivery accessory 16 such as a urinary catheter, intravenous (IV) port, or the like connects the syringe 14 to the patient (note, FIG. 1 diagrammatically indicates the patient accessory 16 by showing a portion of fluid tubing extending away from the syringe 14), and a plunger 18 of the syringe is driven by the syringe infusion pump 10 to deliver a supply of infusion fluid contained in the syringe 14 to the patient at a controlled flow rate. To provide motive force for driving the plunger 18, the syringe infusion pump 10 includes an electric motor 20. (It is noted that the motor 20 is typically an internal component that is disposed within the housing of the infusion pump 10, but is shown outside for illustrative purposes). The electric motor 20 is coupled by gearing or other mechanical hardware (not shown) to drive an arm 22 engaging the plunger 18 of the syringe 14.

The electric motor 20 includes a rotor/stator assembly 24 that drives a rotatable shaft 26 that is coupled with the drive arm 22 of the syringe infusion pump 10 (again, using gearing, clutches, or so forth, not shown; or, more generally, the shaft 26 is operatively mechanically coupled with a component of a medical device or the like that requires operative motive force). The rotor/stator assembly 24 includes a stator comprising electrical windings and a rotor magnetically coupled with the stator to define the electric motor 20. The illustrative motor has a stator that is not electrically driven, and is classified as an induction motor. As disclosed herein, the electric motor 20 does not include ferromagnetic material, and does not include any permanent magnet. The electric motor 20 further includes, or is operatively connected with (e.g. via suitable electrical wires or cable) a motor driver 28 that is operative to electrically power the stator at a fixed electrical frequency.

The syringe infusion pump is disposed inside the MRI room 2, and is shown as an illustrative example of a motorized device that may be usefully used inside the MRI room 2 using an MRI-compatible electric motor 20 as disclosed herein. In other embodiments, the infusion pump may be of a non-syringe variety, in which the fluid delivery component (instead of being the syringe receptacle 12) includes a fluid pump having an inlet configured to connect with an infusion fluid supply (e.g. hanging from an IV stand) and an outlet configured to connect with the patient infusion delivery accessory 16. As another example, a motorized fan may be usefully deployed inside the MRI room 2. Moreover, as previously mentioned an embodiment of an electric motor 20 as disclosed herein may be employed in substantially any other type of motorized device that is used in a setting in which magnetic field interactions may be detrimental to operation of proximate equipment such as a PET imaging device, a SQUID or other magnetometer, or so forth.

The electric motor 20 does not contain any ferromagnetic parts, so it will not be attracted by the magnetic field generated by the MRI device 4. As another advantage, the electric motor 20 does not contain any ferromagnetic parts which might distort the MRI's imaging field. The electric motor 20 generates weak stray fields, which can be designed to be small enough as not to interfere with the MRI's imaging field. Optionally, any remaining stray fields can be shielded using e.g. electrically conductive sheet cover.

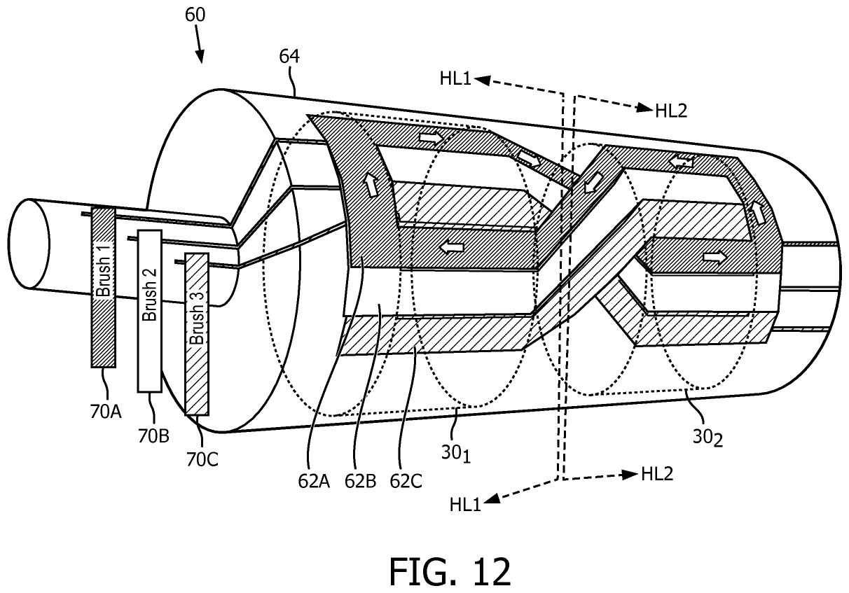

The electric motor 20 is an induction motor. (However, a different type of electric motor is alternatively contemplated, e.g. as illustrated in FIG. 12). However, unlike a conventional induction motor, the electric motor 20 contains no ferromagnetic material (e.g. iron, steel, neodymium, or so forth) in the rotor and stator. In a conventional induction motor, ferromagnetic material is employed to provide a magnetic flux due to the electrical energizing of the stator windings which is many times greater than the magnetic flux produced in the electric motor 20 which does not contain ferromagnetic material. As is known in the art, high magnetic flux provided by the use of ferromagnetic materials enables the achievement of high torque. The omission of ferromagnetic material in the electric motor 20 leads to the following differences compared with a conventional induction motor with ferromagnetic material in its stator and/or rotor: (1) no attraction forces in static magnetic field (such as the Bo magnetic field generated by the MRI device 4); (2) lower efficiency compared to conventional induction motors, because of the omission of a ferromagnetic core for the stator windings; (3) the option of driving at higher frequencies, because the stator coils have lower self-inductance due to the omission of the ferromagnetic core; and (4) the option of driving at constant frequency (no vector control needed), because of a large slip range achievable in the electric motor 20. The lower efficiency of the electric motor 20 as compared with a conventional induction motor with ferromagnetic material is a disadvantage; however, it is recognized herein, and demonstrated via motor characteristics reported herein, that the electric motor 20 can achieve useful torque in spite of its lack of ferromagnetic material.

The coil currents and induced currents produced during operation of the electric motor 20 will generate magnetic fields having the potential to disturb the imaging function of the MRI device 4. However, it is further recognized herein that at normal current levels and realistic distances of the motor from the MRI device (e.g., on the order of a half meter or larger) the fields and field gradients will be low, e.g. fields at or more likely below the milliTesla (mT) range, and gradients at or more likely below the mT/m range. In some illustrative embodiments, an outer sheet rotor is employed, which provides intrinsic shielding and consequent additional reduction of the fields that propagate outside the electric motor 20. Optionally, an additional shielding layer may be applied to further shield the stray fields.

The working principle of an induction motor is that an alternating current through a number of stator coils (typically 3-phase, but other coil distributions exist and are contemplated for the electric motor 20) creates a rotating magnetic field. This rotating magnetic field creates induced currents in the rotor, which in turn create a magnetic field that interacts with the stator field to provide motive force (e.g. torque) causing rotation of the rotor and of the shaft 26 connected to rotate with the rotor. The parts creating the motive force are the electrically conductive parts (coils and rotor). In a conventional induction motor, ferromagnetic material is added to increase the efficiency. However, as disclosed herein, the electric motor 20 does not include ferromagnetic material. With the ferromagnetic material omitted, the electric motor 20 still functions in the same way as a conventional induction motor, although at a significantly lower efficiency.

When the electric motor 20 is operated in a magnetic field environment such as that generated by the operating MRI device 4, there will be several disturbing forces. The external magnetic fields will interact with the currents in the motor coils, creating Lorentz forces. Because the coils of the stator are mechanically connected to a stationary support, this will not cause problems so long as the stator support is sufficient. The external field will also create eddy currents in the electrically conductive material of the rotor, which creates a damping torque proportional to the square of the field and also proportional to the square of the rotation frequency. To counter this effect, a large number of motor coils can be used. This reduces the damping torque because the electrical working frequency is much larger than the rotation frequency of the rotor. Conversely, the motor coils will create magnetic fields which could potentially distort the MRI field. However, because there are multiple coils, their resulting field will decrease very rapidly with distance. Further measures, such as the use of an external sheet rotor as in some embodiments disclosed herein, and/or the use of extra motor shielding, can ensure that the motor's external stray field will stay below the allowed (design-basis) disturbance field.

The induction motor 20 does not include ferromagnetic material. The induction motor 20 (and more particularly the rotor/stator assembly 24) includes a rotor, which may for example comprise a thin-walled electrically conductive cylinder (although a cage-shaped rotor such as a squirrel cage rotor is also contemplated), and a stator comprising a set of coils, e.g. a multiple of three when employing 3-phase input electrical power) arranged at a small distance around or inside the rotor. It is contemplated to exchange the rotating and stationary parts (so that the cylinder is stationary and the coils rotate around or inside it), but this is generally not preferred because this will complicate the electrical connection of the coils.

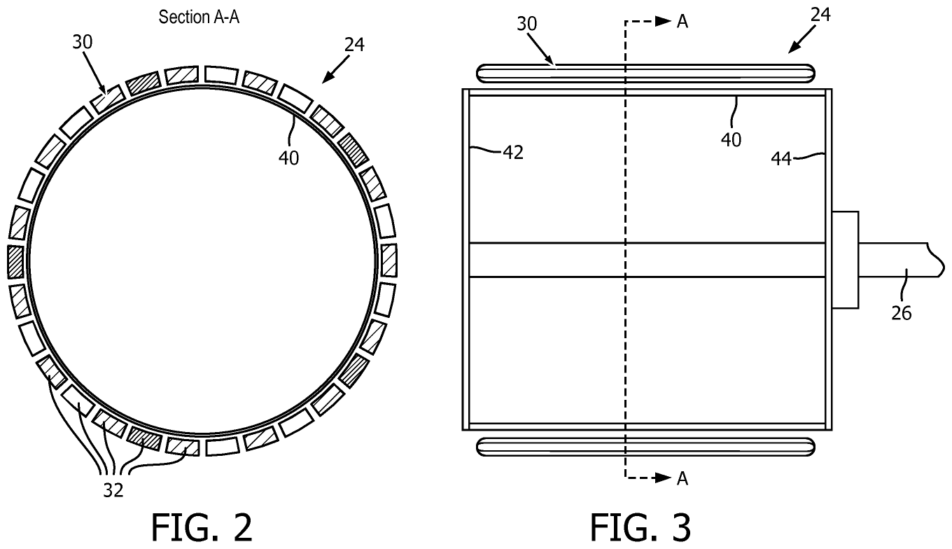

With reference now to FIGS. 2-7, three illustrative embodiments of the rotor/stator assembly 24 are described. Each illustrative embodiment includes a stator 30 which, as best seen in the sectional views of FIGS. 2, 4, and 6, comprises electrical windings 32. The stator 30 is mounted in a fixed fashion (mounting not shown) and receives electrical power to energize the electrical windings 32 from the motor driver 28 (see FIG. 1). The electrical windings 32 are arranged as a pattern of coils with different electrical phases, e.g. three phases repeating in sequentially around the circumference of the stator 30. For example, the electrical windings 32 may be arranged as five sets of 3-phase coils, although a larger or smaller number of sets are contemplated. The electrical frequency of the stator is determined by the electrical frequency of the 3-phase power (e.g. 60 Hz being conventional in the United States, and 50 Hz being conventional in Europe) and the number of sets of 3-phase coils (more sets provides a higher operating electrical frequency for the motor 20). The stator 30 has the same configuration in all three embodiments of FIGS. 2-7; these illustrative embodiments differ by the configuration of the rotor.

With reference to FIGS. 2 and 3, in a first illustrative embodiment, the rotor comprises an inner rotor cylinder 40 disposed inside the stator 30. In this design, the rotor may further include end plates 42, 44 that enclose the ends of the inner rotor cylinder 40, and the shaft 26 extends through to be secured with both end plates 42, 44 to connect the shaft 26 with the rotor. The stator 30 is external to the rotor and hence easily anchored to a stationary support (not shown, e.g. a motor frame).

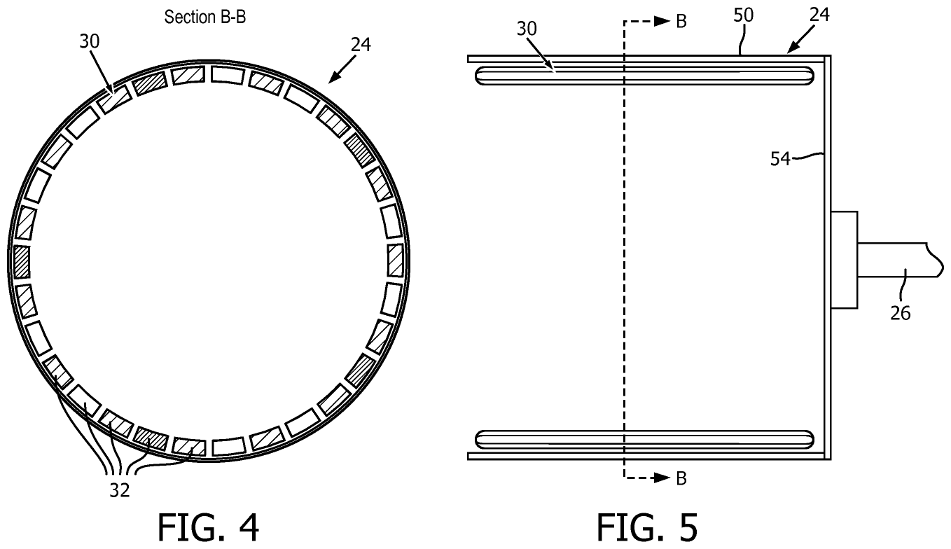

With reference to FIGS. 4 and 5, in a second illustrative embodiment, the rotor comprises an outer rotor cylinder 50 disposed outside the stator 30. The outer rotor cylinder 50 is secured with the shaft 26 at one end by an end plate 54; the end opposite from end plate 54 is open to provide access for anchoring the stator 30 to the stationary support (not shown, e.g. a motor frame).

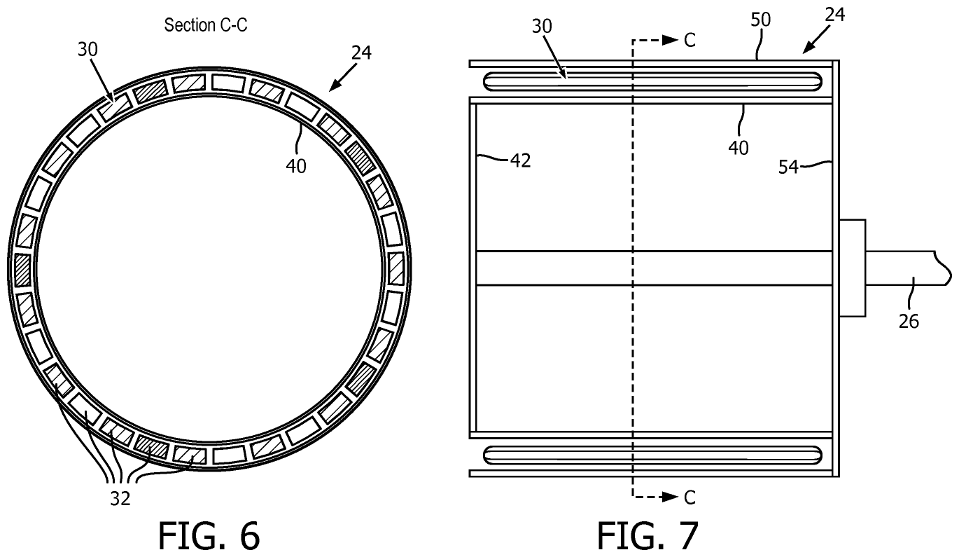

With reference to FIGS. 6 and 7, in a third illustrative embodiment, the rotor comprises both the inner rotor cylinder 40 disposed inside the stator 30 and the outer rotor cylinder 50 disposed outside the stator 30. The end plate 54 provides for securing the inner rotor cylinder 40 and the outer rotor cylinder 50 together so they rotate together to drive the shaft 26. Optionally, the end plate 42 is included as in the first embodiment of FIGS. 2 and 3 in order to provide an additional anchor point for securing the shaft 26 with the rotor.

The embodiments of FIGS. 4-7 which include the outer rotor cylinder 50 disposed outside the stator 30 have a substantial advantage over the embodiment of FIGS. 2-3 which omits this outer rotor cylinder insofar as the outer rotor cylinder 50 provides intrinsic RF and magnetic shielding for the stator 30. This reduces the fields emitted by the motor in the case of the embodiments of FIGS. 4-7, and also reduces the impact of external fields on the motor in these embodiments.

The inner rotor cylinder 40 is, in some embodiments, a cylindrical sheet rotor, that is thin sheet of metal shaped to from the cylinder of the rotor. Likewise, the outer rotor cylinder 50 is, in some embodiments, a cylindrical sheet rotor. This design enhances the shielding provided, especially in the case of an outer cylindrical sheet rotor 50. In other embodiments, the inner and/or outer rotor cylinder 40, 50 may be dielectric cylinder(s), e.g. printed circuit boards (PCBs) with a conductive loop pattern printed or otherwise formed on or in the dielectric cylinder(s). In yet other embodiments, the inner and/or outer rotor cylinder 40, 50 may be squirrel cage rotor(s).

The embodiments of FIGS. 2-7 are illustrative examples, and numerous variants are contemplated. For example, in the embodiments of FIGS. 4-7 including the outer rotor cylinder 50, different arrangements may be employed to provide access to the stator 30 for anchoring it to the motor frame. As another illustrative contemplated variant, different phase schemes are contemplated instead of 3-phase; as long as a rotating magnetic field is created. While the illustrative rotor/stator design is cylindrical, a disc shaped rotor/stator design is alternatively contemplated.

As previously mentioned, it is generally considered necessary in the art to include ferromagnetic material in an induction motor in order to provide sufficient magnetic flux to enable the achievement of high torque. However, it is recognized herein that the disclosed induction motor 20 with no ferromagnetic material can provide sufficient torque for many applications, such as driving an infusion pump, mechanical fan, or so forth.

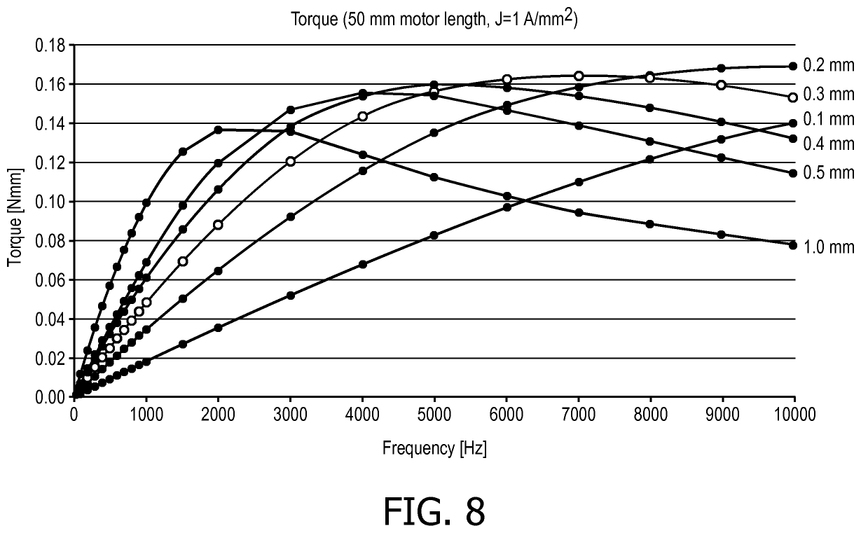

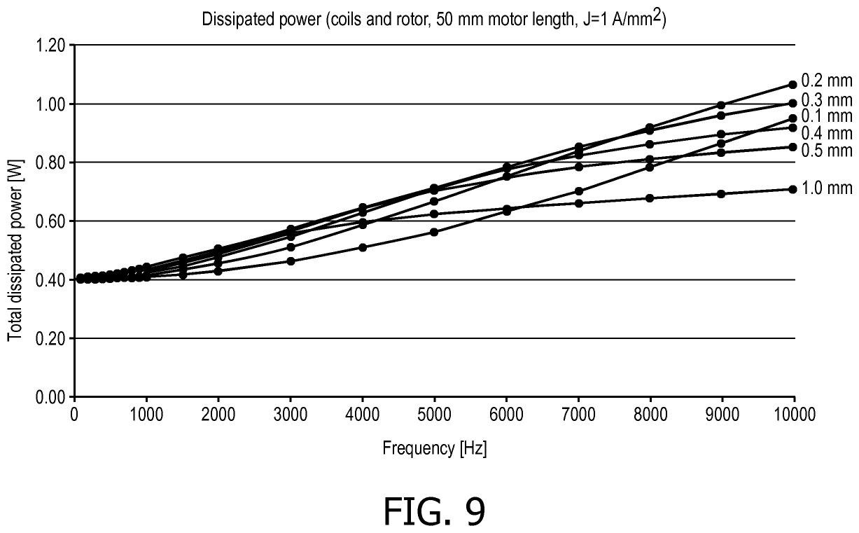

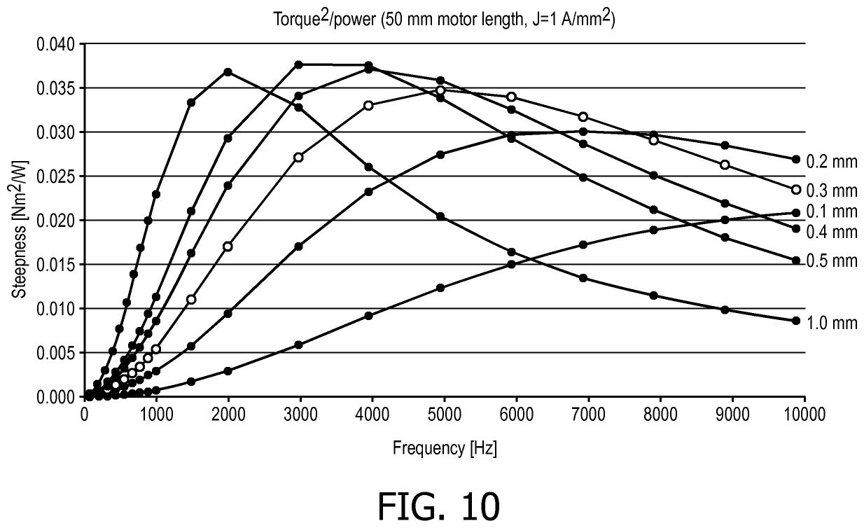

With reference to FIGS. 8-10, calculations of motor characteristics are presented which demonstrate this. For varying frequencies of the 3-phase input current the performance of the motor is calculated. There will be a certain optimum frequency where the torque is maximized, and also an optimum frequency (not necessarily the same) where the motor steepness has a maximum. The motor steepness can be seen as a performance indicator, enabling comparison of efficiencies. The calculations presented in FIGS. 8-10 are for the embodiment of FIGS. 6 and 7 including both inner and outer rotor cylinders 40, 50, and plot motor characteristics of torque (FIG. 8), dissipated power (FIG. 9), and squared-torque/power (FIG. 10) as a function of electrical operating frequency assuming 3-phase power with a number of sets of windings effective to provide the electrical operating frequency shown in the abscissa.

FIG. 8 plots calculated motor torque versus driving frequency for several values of the sheet rotor thickness. From FIG. 8 it can be seen that this illustrative geometry results in 0.14-0.17 N-mm torque at a driving frequency of several kHz. Another salient observation is that, when the number of coils is more than just a few, the rotation frequency of the sheet will result in a slip frequency that is small relative to the driving frequency, so that it is expected to be possible to drive this motor just using one fixed frequency. (That is, the motor driver 28 may optionally be a fixed frequency motor driver operative to electrically power the stator 30 at a fixed electrical frequency). No vector control will be required in that case, which will simplify the electronic driver design of the motor driver 28.

With returning reference to FIG. 1, a method of operating a medical device includes operatively connecting the medical device to a patient, and operating the induction motor 20 to apply motive force to the medical device to deliver a therapy to the patient; where, the induction motor 20 does not include ferromagnetic material and does not include a permanent magnet. Such a method may advantageously further include using the MRI device 4 to acquire MRI images of the patient simultaneously with operating the induction motor 20 to apply the motive force to the medical device to deliver the therapy to the patient. A further advantage is that the induction motor 20 does not rely upon magnetic fields generated by the MRI device 4. Thus, the operating of the induction motor to apply the motive force to the medical device to deliver the therapy to the patient may be repeated when not acquiring MRI images of the patient and with the induction motor 20 located outside of any magnetic field generated by the MRI device (e.g. outside of the MRI room 2). In the illustrative embodiment of FIG. 1, the medical device is an infusion pump 10 and the induction motor 20 is operated to apply pumping force to an infusion fluid to deliver an infusion to the patient. The method may further include, during operation of the induction motor 20, providing electromagnetic shielding of the stator 30 using the rotor (e.g. using the outer rotor cylinder 50). In some such method embodiments, the operating of the induction motor 20 comprises operating the induction motor at a fixed electrical frequency.

The illustrative embodiments of FIGS. 2-7 employ sheet rotors 40, 50. In other embodiments, as previously noted, the inner and/or outer rotor cylinder 40, 50 may be dielectric cylinder(s), e.g. printed circuit boards (PCBs) with a conductive loop pattern printed or otherwise formed on or in the dielectric cylinder(s), or squirrel cage rotor(s).

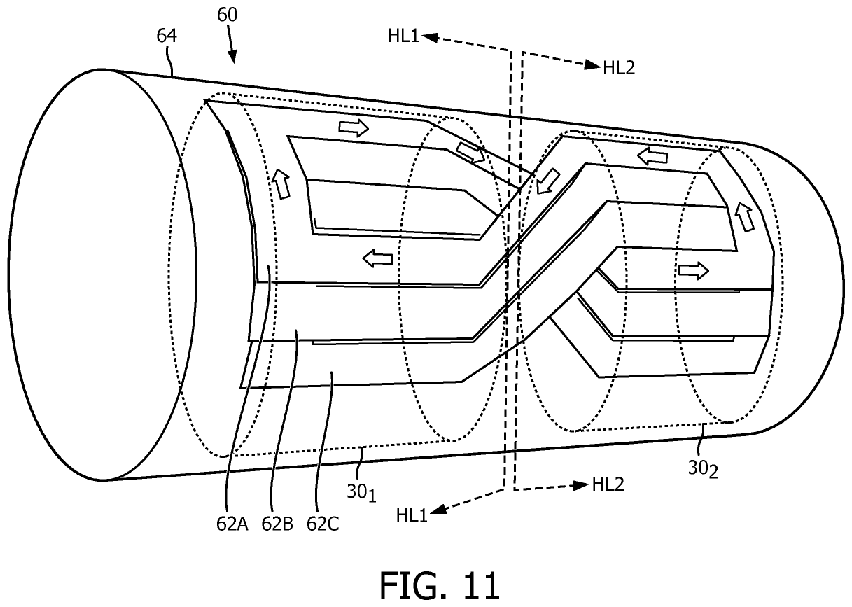

FIG. 11 depicts another illustrative rotor 60, which may be suitably used in place of the inner and/or outer rotor 40, 50. The illustrative rotor 60 includes conductive loop patterns 62A, 62B, 62C disposed on a substrate, e.g. dielectric former, 64 in a three-phase configuration as described below. For example, the rotor 60 may be constructed as a PCB where the substrate 64 is the board of the PCB and the conducting loops 62A, 62B, 62C are implemented as PCB traces.

The magnetic field of the MRI device 4 may induce currents in the conducting parts of the rotor when it is moving, resulting in a damping torque. More particularly, a voltage is induced according to Lenz' law, which results in a current when there is an electrically conductive path. The electrical power dissipated by this current has to be delivered and is added to the mechanical input power of the rotor. Because the mechanical power is expressed as the product of torque and rotation speed, this additional power is observed as a torque proportionally to the rotation speed, so it appears as a pure damping. The magnitude of the induced currents depends on several factors: (i) the magnitude of the magnetic field component that is radially aligned with the rotor; (ii) the rotation speed of the rotor; and (iii) the electrical resistance of the conductive path. Magnetic field components that are axially aligned with the rotor axis will have negligible effect. Therefore, if the rotor is oriented such that the rotor axis is not aligned with the local MRI (stray) field, additional damping will occur. Under unfavorable conditions (high B field, high rotation speed), this additional damping torque may significantly limit the performance of the motor.

To prevent this, the illustrative rotor 60 is not shaped as a closed sheet (that is, not a sheet rotor) but rather comprises one or more conducting loops 62A, 62B, 62C. These loops are shaped such that the induced voltage in one half of the loop (indicated as half-loop HL1) cancels the effect of the induced voltage in the other half-loop HL2. In the illustrative example, this is achieved by the conducting loops 62A, 62B, 62C having a pattern resembling a figure-eight. (At the crossing points the conductors should be isolated from each other, e.g. by using different PCB layers with interposed electrically insulating dielectric layers). Multiple loops can be constructed in this way, such that the rotor is efficiently filled with these conductors. The loops on different layers may overlap each other, provided that they are not connected electrically. The illustrative conducting loops 62A, 62B, 62C are a set of three phases, and each conducting loop comprises a closed contour such that the enclosed areas that have opposite current direction (indicated with arrows only for the conducting loop 62A for illustrative purposes) are equal in size. In contemplated variants, the number of phases may vary, the coil ends can be overlapping in different ways, and/or the loop shape may be varied (while ensuring that the enclosed areas having opposite current direction are equal). A design with more than two loop parts is also contemplated, provided that the sum of all enclosed areas that have clockwise current direction equals the sum of all areas with counterclockwise current direction.

To accommodate the opposing orientations of the loop halves HL1, HL2, the stator is split into two halves, electrically driven at 180 degrees phase difference (generally, at such a phase difference that the induced currents in the loop halves corresponding with the stator excitation are in phase) so that the two loop halves combine their contributions to the torque. In FIG. 11 the split stator is indicated by dashed lines showing a first stator 30₁ magnetically coupled with the first rotor half-loop HL1 and a second stator 302 magnetically coupled with the second rotor half-loop HL2. The illustrative stator 30₁, 30₂ is located inside the rotor 60, i.e. the rotor 60 is an outer rotor as in the embodiment of FIGS. 4 and 5 (but with the sheet rotor 50 replaced by the rotor 60). Although not illustrated, the alternative or additional inner sheet rotor 40 may be similarly replaced by a design corresponding to the rotor 60. In general, rotors of the design of illustrative rotor 60 the conductive paths are shaped in such a way that the effects of the external magnetic field from the MRI device 4 will (at least partially) cancel out, while the effect of the stator currents is maximized by use of the first and second stators 30₁, 30₂ driven with a 180° phase difference.

With reference to FIG. 12, the rotor 60 is again shown. In the arrangement of FIG. 12, the conducting loops 62A, 62B, 62C are connected to respective commutator brushes 70A, 70B, 70C, such that controlled currents can be sent through the conducting loops 62A, 62B, 62C of the rotor via the respective commutator brushes 70A, 70B, 70C. Whereas the motors of the embodiments of FIGS. 2-7 can be classified as induction motors (even with the rotor(s) 40, 50 replaced by the rotor 60 of FIG. 11), the embodiment of FIG. 12 with the conducting loops 62A, 62B, 62C of the rotor 60 driven via the commutators 70A, 70B, 70C is not classified as an induction motor, because it does not make use of induced currents. Some advantages of the embodiment of FIG. 12 are that the rotor currents can be made larger compared to induced currents, and/or the phases of the currents can be controlled in order to achieve optimum torque.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An electric motor comprising:
a stator comprising electrical windings; and
a rotor magnetically coupled with the stator;
wherein the electric motor does not include ferromagnetic material; and
wherein the electric motor does not include any permanent magnet.

2. The electric motor of claim 1, wherein the rotor comprises an outer rotor cylinder surrounding the stator.

3. The electric motor of claim 2, wherein the rotor further comprises an inner rotor cylinder disposed inside the stator and connected to rotate with the outer rotor cylinder.

4. The electric motor of claim 2, wherein the electric motor is an induction motor and the outer rotor cylinder comprises a cylindrical sheet rotor.

5. The electric motor of claim 1, wherein:
the rotor comprises one or more conductive loops each shaped such that the induced voltage in one half-loop (HL1) cancels the effect of the induced voltage in the other half-loop (HL2); and
the stator comprises a first stator magnetically coupled with the one half-loop (HL1) and a second stator magnetically coupled with the other half-loop (HL2), wherein the first and second stators are electrically driven at 180 degrees phase difference.

6. The electric motor of claim 5, further comprising:
a commutator brush operatively coupled with each respective conductive loop.

7. The electric motor of claim 1, wherein the electrical windings of the stator (30) are wound to form the stator as a three-phase stator.

8. The electric motor of claim 1 further comprising:
a fixed frequency motor driver for electrically powering the stator at a fixed electrical frequency.

9. An infusion pump comprising:
an electric motor as set forth in claim 1; and
a fluid delivery component comprising at least one of: (i) a syringe receptacle or (ii) a fluid pump having an inlet configured to connect with an infusion fluid supply; and further comprising an outlet configured to connect with a patient infusion delivery accessory;
wherein the electric motor is connected to operate the fluid delivery component by driving a plunger of an associated syringe mounted in the syringe receptacle or by operating the fluid pump.

10. An infusion pump comprising:
a fluid delivery component comprising at least one of: (i) a syringe receptacle or (ii) a fluid pump having an inlet configured to connect with an infusion fluid supply; and further comprising an outlet configured to connect with a patient infusion delivery accessory; and
an electric motor connected to operate the fluid delivery component by driving a plunger of an associated syringe mounted in the syringe receptacle or by operating the fluid pump;
wherein the electric motor does not include ferromagnetic material and does not include a permanent magnet.

11. The infusion pump of claim 10, wherein the electric motor comprises:
a stator; and
a rotor comprising an outer rotor cylinder surrounding the stator.

12. The infusion pump of claim 9, wherein the rotor further comprises an inner rotor cylinder disposed inside the stator and connected to rotate with the outer rotor cylinder.

13. The infusion pump of claim 11, wherein the electric motor is an induction motor and the outer rotor cylinder comprises a cylindrical sheet rotor.

14. The infusion pump of claim 11 wherein:
the outer rotor cylinder comprises one or more conductive loops, each shaped such that the induced voltage in one loop portion (HL1) cancels the effect of the induced voltage in another loop portion (HL2); and
the stator comprises a first stator magnetically coupled with the one loop portion (HL1) and a second stator magnetically coupled with the other loop portion (HL2), wherein the first and second stators are driven at a phase difference effective to induce currents in the loop halves corresponding with the stator that are in phase.

15. A method of operating a medical device, the method comprising:
operatively connecting the medical device to a patient; and
operating an electric motor to apply motive force to the medical device to deliver a therapy to the patient;
wherein the electric motor does not include ferromagnetic material and does not include a permanent magnet.

16. The method of claim 15, further comprising:
using a magnetic resonance imaging (MRI) device to acquire MRI images of the patient simultaneously with operating the electric motor to apply the motive force to the medical device to deliver the therapy to the patient.

17. The method of claim 16, further comprising:
repeating the operating of the electric motor to apply the motive force to the medical device to deliver the therapy to the patient when not acquiring MRI images of the patient and with the electric motor located outside of any magnetic field generated by the MRI device.

18. The method of claim 15, wherein the medical device is an infusion pump and wherein the electric motor is operated to apply pumping force to deliver an infusion fluid to the patient.

19. The method of claim 15, wherein the electric motor comprises a stator comprising electrical windings and a rotor and the method further comprises:
during operation of the electric motor, providing electromagnetic shielding of the stator using the rotor.

20. The method of claim 15, wherein operating the electric motor comprises operating the induction motor at a fixed electrical frequency.
